(19) United States
(12) Patent Application Publication
(10) Pub. No.: US 2007/0066891 A1
(43) Pub. Date: Mar. 22, 2007

(54) Skin marking for indicating subdermal chip

(76) Inventor: Richard C. Fuisz, McLean, VA (US)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/602,405

(22) Filed: Nov. 21, 2006

Related U.S. Application Data
(63) Continuation of application No. 10/122,299, filed on Apr. 16, 2002, now abandoned.

(51) Int. Cl. A61B 5/05

(52) U.S. Cl. 600/424

(57) ABSTRACT

The presence of a device that is implanted under the skin of a subject and that stores information that can be read by an external device is indicated by an externally visible marking on the skin of the subject.

### CROSS-REFERENCE TO RELATED APPLICATION

This application is a continuation application of U.S. application Ser. No. 10/122,299, filed Apr. 16, 2002, the contents of which are incorporated herein by reference.

### BACKGROUND OF THE INVENTION

The present invention relates to a skin marking to indicate the presence of a subdermal chip.

In recent years, miniaturized chips have been developed that can be implanted under the skin of a subject and that can hold detailed identification data, including complete medical records of the subject. Typically, such chips, once they are implanted, are dormant until activated by an external scanner, at which time they emit a radio frequency signal that can be picked up by the scanner. In operation, an implanted chip may provide hospitals and emergency workers with critical medical information on a subject. For example, if a subject is brought into an emergency room unconscious, or if a subject is an Alzheimer's patient who wanders, the chip may provide everything from basic identification and medical insurance data to vital information about allergies and medications. Moreover, chips may be implanted in pets to provide identification data.

A problem that may arise with implanted chips is that until implanted subdermal chips become more widely used, persons, such as emergency room workers, who could use the information contained in an implanted chip, may be unaware of the chip's presence and may not think to expose the subject to a scanner to read the information. If a subject is unconscious, an Alzheimer's patient or an amnesiac or a nonhuman, the subject will not be able to notify others of the presence of the chip. As smaller chips are developed, it is less likely that there will be scars or other indications that a chip has been implanted. Accordingly, there is a great need for a designation to let others know that a subject has an implanted subdermal chip.

### BRIEF DESCRIPTION OF THE DRAWINGS

FIG. 1 is a schematic cross-sectional view of skin and subdermal regions of a subject having an implanted chip and an external marking in accordance with an embodiment of the present invention.

### DETAILED DESCRIPTION OF THE INVENTION

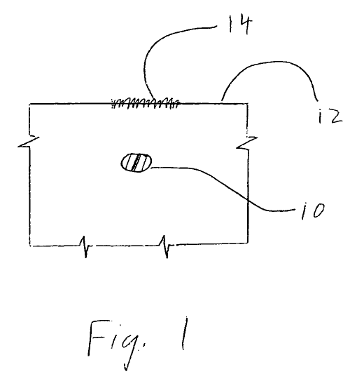

As shown schematically in FIG. 1, a chip 10 is implanted under surface 12 of the skin of a subject. In accordance with the present invention, the surface of the skin includes an external marking 14 that indicates the presence of the subdermal chip.

The subdermal chip in accordance with the present invention can by any device implanted under the skin of a subject to store information in a form that can be read by an external device such as a scanner. For example, an implantable device has been developed by Applied Digital Solutions and marketed under the name of VeriChip. (See, for example, the Applied Digital Solutions Web site at www.adsx.com.) The Verichip device is about the size of a grain of rice and is implanted by way of a needle-like instrument in an outpatient procedure. The chip is activated by an external scanner and emits a radio frequency signal containing a verification number. The verification number is sent to a data storage site to obtain detailed information on the subject. It is anticipated that as the technology develops, such devices will be made even smaller and will contain more detailed information that can be immediately accessed.

The external marking in accordance with the present invention can be any permanent or removable mark imprinted on the surface of the skin of the subject or embedded in the dermal layer of the skin so that it is externally visible or detectable. Preferably, the external marking is m printed or embedded in the skin so that it is not washed or scraped off during normal activities of the subject. For example, the external marking may be in the form of a tattoo, that is, in the form of particles or droplets, such as, for example, ink particles or ink droplets, embedded in the dermal layer of the skin of the subject.

The external marking may range from a simple designation, such as a circle or arrow indicating the presence of the chip, to a more sophisticated written message conveying, for example, information about the make and model of the implanted chip and about the conditions for activating and reading the chip. Standardized numerical or symbolic designations could be developed to be recognized by medical or emergency personnel as a shorthand for conveying technical information about the chip. Preferably, in the development of standardized numerical or symbolic designation, figures that are commonly used in decorative tattoos would be avoided in order to distinguish subjects having decorative tattoos from subjects having external markings in accordance with the present invention.

The process of marking the skin of the subject may be carried out at or about the same time as the implantation of the subdermal device, or may be carried out at a different time, before or after the implantation.

Preferably, the external marking is on the surface of the skin right above or immediately adjacent to the subdermal device, so that the location of the subdermal device can be immediately determined. However, if a subdermal device is of such a type that it is not necessary for the external scanner to be placed immediately above the subdermal device, a precise indication of the location of the subdermal device may not be necessary. In such a case, the external marking may be placed anywhere that it may be reasonably noticeable by emergency or medical personnel.

Obviously, many modifications and variations of the present invention are possible in light of the above teachings. It is therefore to be understood that, within the scope of the appended claims, the invention may be practiced otherwise than as specifically described.

What is claimed is:

1. A combination comprising
an implanted device implanted under the skin of a subject, the implanted device storing information of the subject and being dominant until read by an external device, the information being selected from the group consisting of identification data, medical records, insurance information, subject allergies and subject medications, and
a marking that is externally visible on the skin of the subject and that designates the presence of the implanted device.

2. The combination of claim 1, wherein the information stored on the device is identification data comprising a verification number corresponding to detailed information on the subject stored in a separate data storage site.

3. The combination of claim 1, wherein the information stored on the device consists of medical records.

4. The combination of claim 1, wherein the information stored on the device includes insurance information.

5. The combination of claim 1, wherein the information stored on the device includes subject allergies.

6. The combination of claim 1, wherein the information stored on the device includes subject medications.

7. The combination of claim 1, wherein the externally visible marking is in the form of a tattoo.

8. The combination of claim 1, wherein the externally visible marking is located at or adjacent to a subdermal location of the implanted device so that the externally visible marking indicates the location of the implanted device.

9. The combination of claim 1, wherein the externally visible marking conveys information regarding the identity of the implanted device.

10. A method of indicating the presence of a device that is implanted under the skin of a subject and that stores information that can be read by an external device, the method comprising the steps of
providing a subject having an implanted device that is implanted under the skin of a subject and that stores information of the subject that can be read by an external device, the information being selected from the group consisting of identification data, medical records, insurance information, subject allergies and subject medications;
creating a marking that is externally visible on the skin of the subject to indicate the presence of the implanted device; and
activating the implanted device with an external device to enable the implanted device to transfer the stored information to the external device.

11. The method of claim 10, wherein the step of creating an externally visible marking on the skin of the subject is carried out by creating a tattoo on the skin of the subject.

12. The method of claim 10, wherein the information stored on the device is identification data comprising a verification number corresponding to detailed information on the subject stored in a separate data storage site.

13. The method of claim 10, wherein the information stored on the device consists of medical records.

14. The method of claim 10, wherein the information stored on the device includes insurance information.

15. The method of claim 10, wherein the information stored on the device includes subject allergies.

16. The method of claim 10, wherein the information stored on the device includes subject medications.

17. The method of claim 10, wherein the implanted device remains dormant until activated by the external device.

18. The method of claim 10, wherein the externally visible marking is located at or adjacent to a subdermal location of the implanted device so that the externally visible marking indicates the location of the implanted device.

19. The method of claim 10, wherein the externally visible marking conveys information regarding the identity of the implanted device.
